(19) United States
(12) Patent Application Publication
(10) Pub. No.: US 2010/0185268 A1
(43) Pub. Date: Jul. 22, 2010

(54) IMPLANTABLE MEDICAL DEVICES AND ASSOCIATED SYSTEMS AND METHODS

(75) Inventors: Brad C. Fowler, Duvall, WA (US); Jay Miazga, Seattle, WA (US); Matt L. Bielstein, Renton, WA (US)

Correspondence Address: St. Jude Medical Neuromodulation Division, 6901 Preston Road, Plano, TX, 75024, US

(73) Assignee: Northstar Neuroscience, Inc., Seattle, WA (US)

(21) Appl. No.: 12/414,626

(22) Filed: Mar. 30, 2009

Related U.S. Application Data
(60) Provisional application No. 61/145,482, filed on Jan. 16, 2009.

(51) Int. Cl. A61N 1/05

(52) U.S. Cl. 607/116

(57) ABSTRACT

Implantable medical devices and associated systems and methods are disclosed. An implantable device in accordance with one embodiment can include a signal generator positioned to be implanted in a patient. The signal generator includes a housing and a plurality of selectively electrically activatable portions at an external surface of the housing. The implantable device can also include a remote electrode device having at least one electrode positioned to be implanted beneath the patient's skull, and a lead coupleable to the electrode device and the signal generator.

### CROSS-REFERENCE TO RELATED APPLICATION

This application claims the benefit of U.S. Provisional Patent Application No. 61/145,482, filed Jan. 16, 2009, which is incorporated herein in its entirety.

### TECHNICAL FIELD

The present disclosure relates generally to implantable medical devices and associated systems and methods.

### BACKGROUND

Many patient devices include control systems that are implanted in the patient. Electrical stimulation of neural and cardiac tissue typically involves the use of systems including an implanted pulse generator (IPG) connected to an electrode lead. The electrode is placed over a specific target region in a patient's brain or heart, and the IPG is usually implanted in a subclavicular pocket created beneath the patient's shoulder. Electrical stimulation can be administered using bipolar stimulation or unipolar/monopolar stimulation. Bipolar stimulation is directed to activation of the electrical contacts in the electrode with both anodic and cathodic polarities. In contrast, monopolar stimulation is directed to activation of the electrical contacts in the electrode with one polarity (either anodic or cathodic), and the IPG is configured with the other polarity. In other words, current flows between pair(s) of electrodes or electrical contacts that reside at a stimulation site in the bipolar configuration, and flows between the electrode(s) and the IPG in the monopolar configuration.

When a neuro- or cardio-stimulation device is activated in the monopolar configuration, current passes through patient tissue and fluids between the electrode and IPG. The IPG is typically implanted in subcutaneous connective tissue between the skin and underlying muscle. This connective tissue contains somatosensory nerves and nerve endings that can be activated by the electrical currents along a current path between the electrode and the IPG and, in some cases, generate buzzing or tingling sensations felt by the patient. Muscle tissue adjacent to the IPG can also be activated by these electrical currents. In both cases, the somatosensory or muscle tissues are generally only activated when in very close proximity to the IPG due to the sharp decline of current intensity with distance from the IPG.

Conventional IPGs configured to provide monopolar stimulation are often coated on one side with a non-conductive material. This non-conducting side is implanted face down (i.e., away from the patient's skin) to avoid activation of the underlying muscles, since constantly twitching muscles (e.g., in the shoulder) may be bothersome to the patient. This arrangement, however, reduces the surface area available for passage of current into the IPG and, therefore, increases current intensity across the remaining uninsulated IPG surface that faces up toward the patient's skin. Patients have reported tingling or buzzing sensations around an IPG, particularly when it is coated on one side with an insulator, if the current amplitude is set at moderate to high levels of intensity. If the IPG is active for extended periods of time, this may become annoying or irritating for the patient. Implantation of an uncoated IPG can reduce the current intensity by significantly increasing (e.g., two times as much or more) the exposed IPG surface for passage of current. Such uncoated IPGs, however, can increase the possibility of muscle twitching within the patient, which may more of a problem than the tingling or buzzing sensation associated with activation of somatosensory fibers or nerve endings.

### BRIEF DESCRIPTION OF THE DRAWINGS

FIG. 1 is a partially schematic, isometric illustration of a patient having an implanted medical device configured in accordance with several embodiments of the disclosure.

FIG. 2 is a partially schematic illustration of an implantable pulse system configured in accordance with several embodiments of the disclosure.

FIG. 3 is a partially schematic illustration of an implantable device configured in accordance with an embodiment of the disclosure.

FIG. 4 is a partially schematic illustration of an implantable device configured in accordance with another embodiment of the disclosure.

FIGS. 5A-5C are partially schematic illustrations of an implantable device configured in accordance with still another embodiment of the disclosure.

FIGS. 6A and 6B are partially schematic illustrations of portions of implantable devices configured in accordance with several embodiments of the disclosure.

FIGS. 7A and 7B are partially schematic illustrations of implantable devices configured in accordance with still further embodiments of the disclosure.

FIGS. 8A and 8B illustrate an implantable device configured in accordance with yet another embodiment of the disclosure.

FIG. 9 is a schematic illustration of an implantable device configured in accordance with still another embodiment of the disclosure.

FIG. 10 is a schematic illustration of an implantable device configured in accordance with yet another embodiment of the disclosure.

FIGS. 11A and 11B are partially schematic illustrations of an implantable device configured in accordance with still another embodiment of the disclosure.

FIGS. 12A and 12B are partially schematic illustrations of an implantable device configured in accordance with still yet another embodiment of the disclosure.

### DETAILED DESCRIPTION

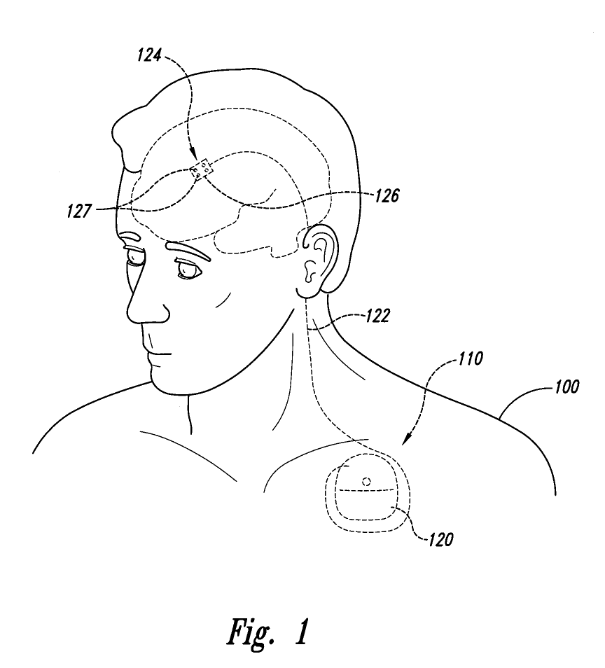

Aspects of the present disclosure are directed generally to implantable medical devices and associated methods for controlling such implantable medical devices. Several details describing structures and processes that are well known and often associated with such systems and methods are not set forth in the following description for purposes of brevity. Moreover, although the following disclosure sets forth several representative embodiments of implantable devices and associated systems, several other embodiments can have different configurations and/or different components than those described in this section. Accordingly, such embodiments may include additional elements and/or may eliminate one or more of the elements described below with reference to FIGS. 1-12B.

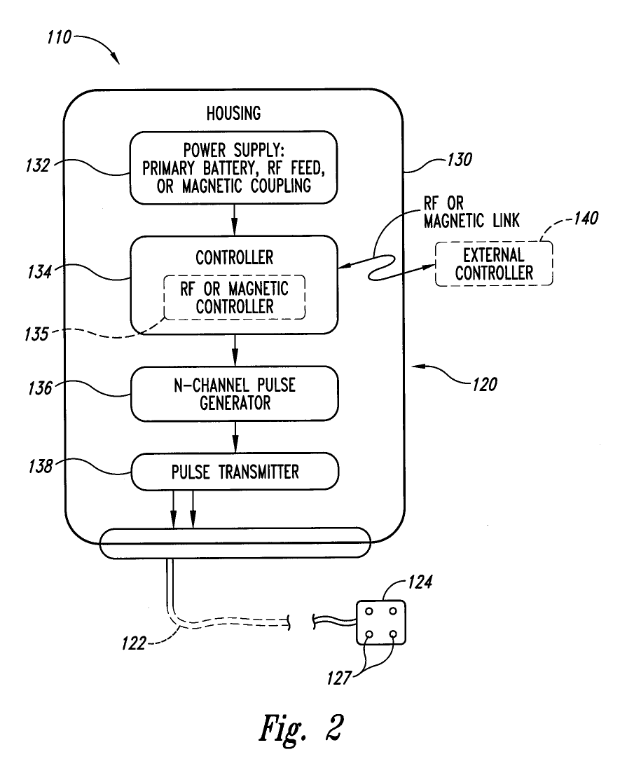

FIG. 1 is a partially schematic, isometric illustration of a patient 100 with an implanted signal delivery system 110 configured in accordance with several embodiments of the disclosure. The system 110 includes an IPG 120 (shown schematically) coupled to one or more signal delivery devices 124 (e.g., electrodes) with a lead 122. The electrode 124 can in turn include a support member 126 carrying one or more electrical contacts 127. The electrode 124 is placed beneath or within the patient's skull. The IPG 120, which provides electrical pulses to the electrode 124, is generally placed below the patient's clavicle. In other embodiments, however, the IPG 120 may be positioned at other suitable locations. Compared to conventional IPGs that can deliver monopolar stimulation, the IPG 120 is configured to prevent, inhibit, or reduce the likelihood of so-called “pocket stimulation” around the implantable device. Pocket stimulation generally occurs in systems using monopolar stimulation because of a concentration of current density or flow at or near the IPG (i.e., the anode or cathode). As used herein, the term “pocket stimulation” includes tingling or buzzing sensations associated with the activation of somatosensory fibers or nerve endings, muscle twitching, or other similar motor and/or sensory responses within the patient's tissue proximate to the implantable device. Although pocket stimulation is generally not dangerous, it can be uncomfortable and/or distracting to the patient. Furthermore, several embodiments of the IPG 120 are also configured to more closely conform to the anatomy of an implant site of the patient. This arrangement is expected to help increase patient comfort and be cosmetically more acceptable than many conventional, relatively bulky implantable devices. Various specific details of embodiments of representative IPGs and associated systems and methods are described in detail below with reference to FIGS. 2-12B.

FIG. 2 schematically illustrates details of the system 110 described above. The IPG 120, for example, generally includes a number of components carried by or contained in a housing 130. The IPG 120 can include a power supply 132, an integrated controller 134, a pulse generator 136, and a pulse transmitter 138 in the housing 130. The housing 130 of the IPG 120 is often referred to as the “can” or “can electrode” and may include a signal return electrode. As described in greater detail below, for example, one or more portions of the housing 130 can be selectively configured to act as the return electrode in monopolar configurations.

The power supply 132 can include a primary battery, such as a rechargeable battery, or other suitable device for storing electrical energy (e.g., a capacitor or supercapacitor). In other embodiments, the power supply 132 can include an RF transducer or a magnetic transducer that receives broadcast energy emitted from an external power source and that converts the broadcast energy into power for the electrical components of the IPG 120 and the other components of the system 110.

In one embodiment, the integrated controller 134 can include a processor, a memory, and/or a programmable computer medium. The integrated controller 134, for example, can be a microcomputer, and the programmable computer medium can include software loaded into the memory of the computer, and/or hardware that performs the requisite control functions. In another embodiment identified by dashed lines in FIG. 2, the integrated controller 134 can include an integrated RF or magnetic controller 135 that communicates with an external controller 140 via an RF or magnetic link. In such an embodiment, the external controller 140 is external of the patient and many of the functions performed by the integrated controller 134 may be resident on the external controller 140 and the integrated portion 135 of the integrated controller 134 may include a wireless communication system.

The integrated controller 134 is operatively coupled to, and provides control signals to, the pulse generator 136, which may include a plurality of channels that send appropriate electrical pulses to the pulse transmitter 138. The pulse transmitter 138 is coupled to one or more signal delivery devices or electrodes 124 (only one is shown). In one embodiment, each electrode is configured to be physically connected to a separate lead, allowing each electrode 124 to communicate with the pulse generator 136 via a dedicated channel. Accordingly, the pulse generator 136 may have multiple channels, with at least one channel associated with each of the electrodes 124. Additionally, or in lieu of the foregoing arrangement, individual electrode contacts 127 carried by an electrode 124 can be individually addressable. Suitable components for the power supply 132, the integrated controller 134, the external controller 140, the pulse generator 136, and the pulse transmitter 138 are known to persons skilled in the art of implantable medical devices.

The system 110 can be programmed and operated to adjust a wide variety of stimulation parameters, for example, which electrodes 124 are active and inactive, whether electrical stimulation is provided in a monopolar (unipolar) or bipolar manner, signal polarity, and/or how stimulation signals are varied. In particular embodiments, the system 110 can be used to control the polarity, frequency, duty cycle, amplitude, and/or spatial and/or topographical qualities of the stimulation. Representative signal parameter ranges include a frequency range of from about 0.5 Hz to about 125 Hz, a current range of from about 0.5 mA to about 15 mA, a voltage range of from about 0.25 volts to about 10 volts, and a first pulse width range of from about 10 μsec to about 500 μsec The stimulation can be varied to match, approximate, or simulate naturally occurring burst patterns (e.g., theta-burst and/or other types of burst stimulation), and/or the stimulation can be varied in a predetermined, pseudorandom, and/or other aperiodic manner at one or more times and/or locations. The signals can be delivered automatically, once initiated by a practitioner. The practitioner (and, optionally, the patient) can override the automated signal delivery to adjust, start, and/or stop signal delivery on demand. The stimulation signals can be selected to have an inhibitory, facilitatory (e.g., excitatory), and/or plasticity-enhancing or facilitating effect on a target neural population to which the signals are directed.

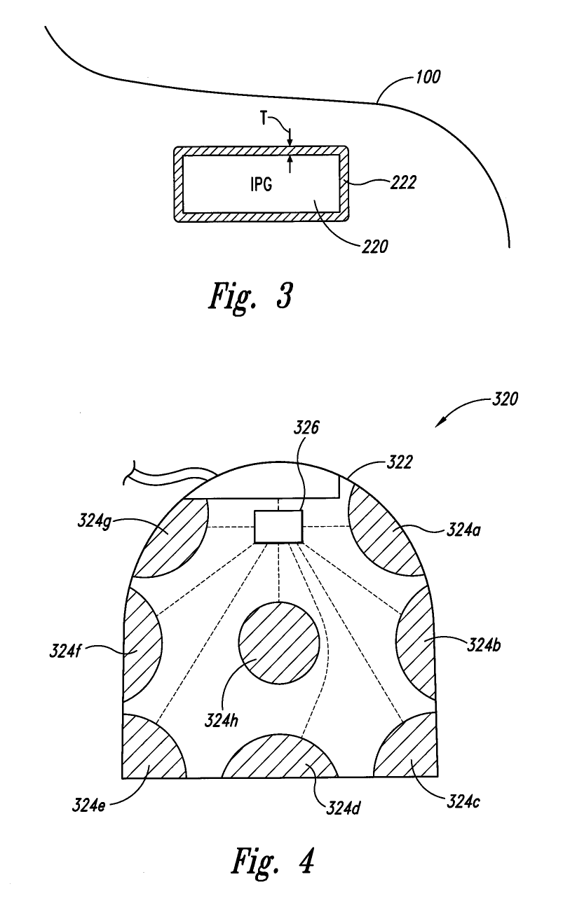

FIGS. 3-12B illustrate various embodiments of IPGs suitable for use with implanted signal delivery systems (e.g., the system 110 described above with reference to FIGS. 1 and 2). The IPGs described below can include many or all of the features and components described above with reference to FIGS. 1 and 2. FIG. 3, for example, is an enlarged schematic view of an IPG 220 configured in accordance with an embodiment of the disclosure. In this embodiment, the IPG 220 includes a sheath or covering 222 carrying the IPG 220. The sheath 222 at least approximately completely surrounds the IPG 220 and has a thickness T that spaces the IPG 220 apart from the surrounding tissue of the patient 100. The sheath 222 can be an integral component of the IPG 220 or a separate component (e.g., a pouch) into which the IPG 220 is placed. It is important to note that that the sheath 222 allows the flow of current into or out of the IPG 220 and, accordingly, the sheath 222 should be electrically conductive.

The thickness T of the sheath 222 can vary from about 0.5 mm to about 5 mm around the conductive surface area of the IPG depending on the total surface area of the IPG 220 that is available for passage of electrical current and the intensity of current used to treat the patient 100. In some instances, the thickness T necessary to prevent activation of overlying somatosensory and/or motor nerves in the patient 100 can be reduced by avoiding the use of non-conductive coatings on the underlying IPG surface since this increases the overall area of the IPG 220 available for current passage.

The sheath 222 may be composed of implantable materials that are resorbable, non-resorbable, nondegradable, and/or absorbable. Resorbable materials include materials that are actively resorbed by inflammatory and other cells in the body that break down and phagocytose the material over time. These materials are commonly used as hemostatic materials in surgery and as wound dressings on the skin and include, for example, collagen, alginate, cellulose, and combinations of these materials. The resorption rate of these materials can be controlled by manufacturing processes which, in general, either retain or break down the structural integrity of the material, making it more or less resistant to resorption by the body. In the extreme, selection of the material and/or its processing can make the material relatively resistant to degradation over the lifetime of an IPG implant, thus making it non-resorbable. Non-degradable materials are also in general use for surgical procedures, and are intended for permanent implants. These materials include, for example, Dacron polyethylene terepthalate fibers that are woven or knitted into sheets, tubes, and other shapes depending on their intended use. Expanded PTFE or Gore-tex is another material used for permanent implants to make vascular grafts, sheets for hernia repair, etc. Absorbable materials are made from water soluble constituents, such as polysaccharides, which dissolve over time and dissipate into the interstitial fluids of the body.

The sheath 222 can be composed of any of the materials described above or other suitable materials. In the case of porous resorbable materials (e.g., collagen), it is well known that inflammatory cell migration (e.g., neutrophills and other leukocytes, and macrophages which can also form multinucleated foreign body giant cells) and, subsequently, connective tissue infiltration (e.g., fibroblasts that deposit a collagen extracellular matrix and capillary ingrowth) occurs within the material porosity. Thus, as the resorbable material is removed by inflammatory cells, connective tissue can replace it and maintain the “space” or “gap” required to prevent and/or inhibit pocket stimulation around the IPG 220, even after the original implanted material is gone. In the case of a sheath composed of non-resorbable materials like Dacron or Gore-tex, the material remains until the IPG is surgically removed. The porous nature of these non-resorbable materials that typically allow tissue ingrowth, however, would require surgical dissection from surrounding tissues for extraction with the IPG 220. In the case of absorbable materials, the sheath 222 would dissolve over time and would not typically be replaced by new connective tissue. In this instance, the protective “space” around the IPG 220 would likely be diminished and eventually allow pocket stimulation effects to develop in the patient 100.

As discussed previously, the sheath 222 should also accommodate the passage of electrical current between the tissue of the patient 100 and the IPG 220. Accordingly, in several embodiments the sheath 222 may include porous material(s) that become filled with saline (applied during implantation for example) or by permeation of interstitial fluids or blood after implantation. Alternatively, the material of which the sheath 222 is composed can be filled with an electrically conductive material in the manufacturing process and can be provided to the surgeon ready for implantation and electrical conduction.

The shape and/or configuration of the sheath 222 can vary depending on the configuration of the IPG 220. IPGs are generally manufactured in a variety of different shapes and/or sizes depending on the desired use, and the sheath 222 can be tailored to fit snugly around a specific IPG for easy insertion into the subcutaneous pocket of a patient. In other embodiments, however, the sheath 222 can be configured to fit a range of different IPG shapes and sizes. In still other embodiments, a number of sheaths 222 can be configured to accommodate various ranges of IPGs.

In any of these embodiments, the thickness of the sheath material should be designed to create sufficient space around the IPG 220 to prevent activation of adjacent nerves and/or muscles in the patient. As discussed above, this thickness can vary between a fraction of a millimeter to several millimeters depending on the conductive IPG surface and intended current intensity. Another particular aspect of the sheath material thickness is how much the selected material compresses when placed into the subcutaneous pocket. Because many of the materials described above are porous to allow permeation of fluids (initially) and cells (over the duration of implant time), these pores can compress when placed into the subcutaneous tissue. Thus, the “space” as defined in this disclosure refers to the distance between the IPG 220 and the surrounding tissue of the patient after implantation and subsequent compression of the material of the sheath 222.

One feature of the embodiment described above with respect to FIG. 3 is that the sheath 222 can include a resorbable or biodegradable material. If the sheath 222 is composed of such materials, it can eliminate the need to excise the IPG 220 after treatment. For example, certain neuromodulation applications only require an implantable medical device for a finite period of time. In these circumstances, a biodegradable pouch material (e.g., collagen) would avoid the need for excising the pouch since it would eventually be resorbed by the body. In contrast, many implantable medical devices remain implanted indefinitely. Cardiac pacemakers, for example, tend to be permanently implanted for the remainder of the patient's life. Sheaths or housings for these devices are generally composed of non-degradable materials, such as a “Parsonnet” pouch (i.e., an IPG or pacemaker pouch composed of a Dacron material). The Parsonnet pouch was developed to prevent problems in patients with “twiddler's syndrome” (e.g., cardiac patients who manipulate their IPG or pacemaker, which can lead to IPG movement or electrode damage). The Dacron material of the pouch is stretchy so that it can accommodate different sizes of IPG and porous so that connective tissue can grow into the pores to fix the material to surrounding tissues. The Dacron material is non-degradable and can remain implanted permanently. However, if it becomes necessary to remove the Dacron pouch from a patient (e.g., because of an infection within the pores of the Dacron material), the pouch will need to be surgically excised since tissue incorporation into the material porosity will prevent easy retrieval. In many neuromodulation applications where an IPG is not necessarily intended to be a permanent implant, the use of a pouch composed of Dacron or other non-degradable materials may be contraindicated.

FIG. 4 is a partially schematic illustration of an IPG 320 configured in accordance with another embodiment of the disclosure. The IPG 320 includes a housing 322 with a plurality of programmable or selectable regions 324 (eight are shown as regions 324a-h). The housing 322 can be composed of an electrically insulating material and/or coated with a non-conductive material. The programmable regions 324a-h can be composed of a conductive material. The programmable regions 324a-h are “hot spots,” and one or more of the regions 324a-h can be electrically activated to provide a set of electrical current return pathways during treatment signal delivery operations. In some embodiments, the overall exposed surface area of the programmable regions 324a-h is nearly the same as the area of the IPG housing 322, such that the aggregate surface area of the nonconductive spaces between the programmable regions 324a-h is relatively small (e.g., in one embodiment, the area of the nonconductive gaps, borders, boundaries, or regions of the housing 322 is less than the average area of each programmable region 324a-h, or less than the area of the smallest programmable region 324a-h). In other embodiments, the spacing or separation between programmable regions 324a-h can be larger, and can be defined based upon patient physiology (e.g., in accordance with expected nerve pathway locations). In the illustrated embodiment, the programmable regions 324a-h are individually addressable. In other embodiments, however, two or more of the programmable regions 324a-h can be linked. The programmable regions 324a-h can be selectively activated/deactivated using hardware and/or software switches. For example, the IPG 320 can include a software switch 326 (shown schematically) that responds to commands, pulse trains or other signals to toggle through a selection of regions 324a-h. In other embodiments, the IPG 320 can include other suitable switching mechanisms to selectively activate/deactivate the regions 324a-h. Depending upon embodiment details, one or more of the regions 324a-h can be activated/deactivated in a predetermined (e.g., activated one-time, or switched at particular times) or aperiodic (e.g., pseudorandom) manner.

As discussed previously, conventional monopolar stimulation arrangements use the “can” or housing of the IPG as one of the electrodes (i.e., anode or cathode). Further, the side of the IPG that faces the muscle (i.e., faces posteriorly) is generally coated to provide insulation. This coating, however, is not always effective at eliminating pocket stimulation in the patient. One particular aspect of the IPG 320 is that one or more of the regions can be selectively activated during treatment, rather than using the entire housing 322 as the return electrode. In one embodiment, for example, all of the regions 324a-h can be activated during treatment. If pocket stimulation occurs, one or more of the programmable regions 324a-h can be deactivated or turned off until the pocket stimulation subsides or is eliminated. Moreover, one advantage of the IPG 320 is that only a small region of the can located near sensitive motor and/or sensory neurons needs to be turned “off,” allowing a relatively large surface area of the can to remain active during treatment.

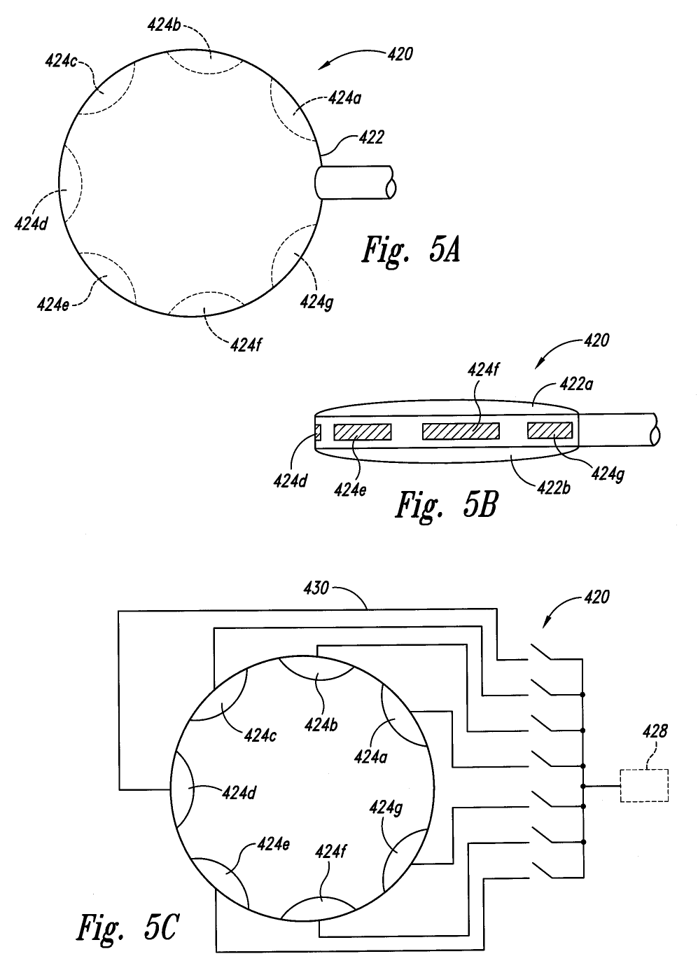

FIGS. 5A-5C are partially schematic views of an IPG 420 configured in accordance with still another embodiment of the disclosure. FIG. 5A, for example, is top plan view and FIG. 5B is a side view of the IPG 420. Referring to FIGS. 5A and 5B together, the IPG 420 includes a housing 422 having a first or upper portion 422a and a second or lower portion 422b. The IPG 420 also includes a plurality of programmable regions 424 (seven are shown as regions 424a-g) at a periphery or edge 423 of the IPG 420 between the first and second housing portions 422a and 422b. The first and second housing portion 422a and 422b can be composed of an electrically insulating material, and the programmable regions 424a-g can be composed of a conductive material. The IPG 420 differs from the IPG 320 described above with reference to FIG. 4 in that the programmable regions 424a-g of the IPG 420 are arranged around the periphery 423 of the IPG 420. In contrast, the programmable regions 324a-h of the IPG 320 are at the major surface of the IPG 320 rather than an edge or periphery of the IPG 320.

The programmable regions 424a-g can be generally similar to the regions 324a-h described above. For example, the regions 424a-g can be individually activated/deactivated during treatment using hardware and/or software switches. FIG. 5C, for example, is a schematic view of the IPG 420 including a software controlled switch 428 that selectively switches one or more of the regions 424a-g on or off during treatment using links 430. The switch can be located external to the IPG 420 or the switch can be integral with the IPG 420. In some embodiments, for example, it may be desirable to have the switch integral with the IPG 420 to minimize the number of external leads projecting from the IPG 420. In other embodiments, the IPG 420 can have a different switching arrangement, e.g., an arrangement in which pulses or other signal modulations are used to selectively activate or deactivate individual regions 424a-g.

In operation, the IPGs 320 and 420 described above with reference to FIGS. 4-5C are generally implanted in a patient with all of the programmable regions 324a-h and 424a-g active. It is generally desirable to have the maximum number of regions active during treatment without causing pocket stimulation in the patient in order to keep the current density low. If the patient experiences pocket stimulation during treatment, however, one or more of the regions 324a-h and 424a-g can be selectively turned off until the sensation/motor response goes away.

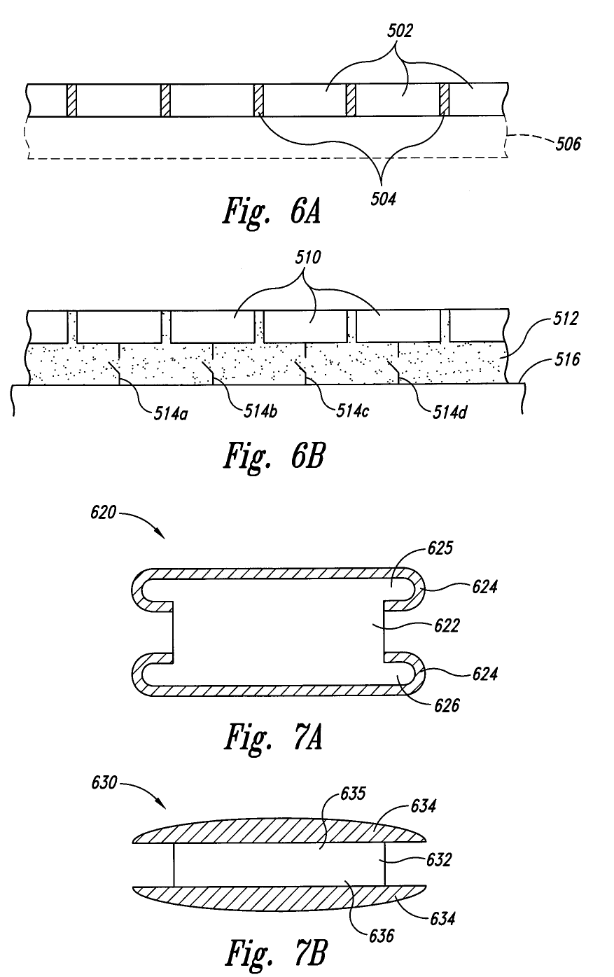

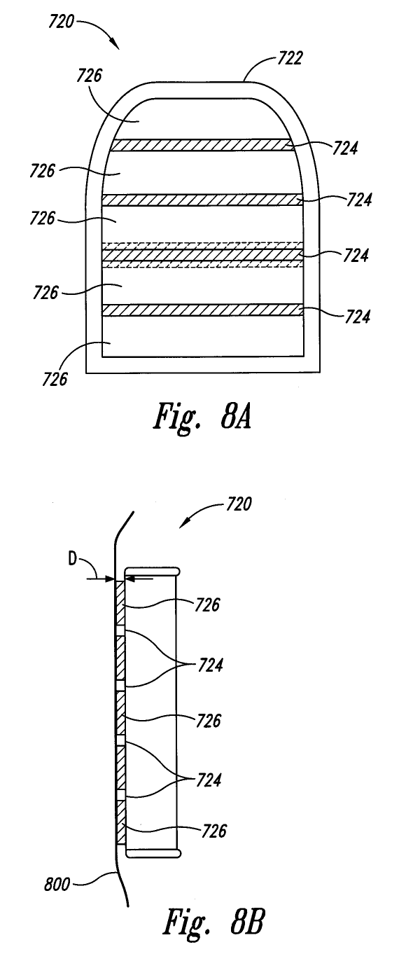

In various embodiments, the programmable regions 324a-h and 424a-g described above with reference to FIGS. 4-5C are generally composed of a plastic and/or ceramic layer with metal (conductive) portions carried by or attached in a desired arrangement to the ceramic or plastic layer. FIGS. 6A and 6B, for example, are partially schematic, side cross-sectional views illustrating various configurations of the programmable regions. The embodiments shown in FIGS. 6A and 6B can be used in the IPGs 320 and 420 described above, or in other suitable IPGs. In the embodiment shown in FIG. 6A, for example, a plurality of conductive (e.g., metal) portions 502 are separated from each other by insulating portions 504. The conductive portions 502 and insulating portions 504 can be carried by a support member 506 (shown in broken lines) composed of plastic or another suitable material at an external surface of an IPG (e.g., the IPG 320 or the IPG 420).

FIG. 6B is a partially schematic illustration of another embodiment in which the individual conductive portions are selectively switchable. More specifically, the arrangement of FIG. 6B includes a plurality of conductive (e.g., metal) portions 510 separated from each other by an insulating material 512. A plurality of switches 514 (four are shown as switches 514a-d) are carried by an external surface 516 of the IPG and positioned to selectively activate/deactivate the respective conductive portions 510. In other embodiments, the programmable regions of the IPGs 320 and 420 can have other configurations and/or include different features.

FIGS. 7A and 7B are partially schematic views of IPGs configured in accordance with still further embodiments of the disclosure. FIG. 7A, for example, illustrates an IPG 620 including a housing or “can” 622 and a coating or insulating layer 624 (e.g., a parylene material) disposed over at least a first or upper portion 625 of the housing 622 and a second or lower portion 626 of the housing 622. FIG. 7B illustrates an IPG 630 having a different configuration than the IPG 620. The IPG 630 of FIG. 7B includes a housing 632 and a coating or insulating layer 634 deposited over an upper and a lower portion 635 and 636 of the housing 632.

In each embodiment, the insulating layers 624 and 634 overhang the edges of the respective housings 622 and 623, and the only portion that remains uncoated is a transition region between the upper and lower portions of the respective housings 622 and 632. One aspect of this arrangement of the insulating layers 624 and 634 is that it can help prevent and/or inhibit “corner effects” in the IPG. For example, as discussed previously, pocket stimulation primarily results from a concentration of current density at a level sufficient to activate a patient's nerve. If the current path to the IPG is primarily via the vasculature, the current may approach the IPG through a blood vessel and then exit the vessel near the IPG and pass through the interlaying connective tissue. This could create a concentration of current density as the current exits the blood vessel, especially if there is a bend or bifurcation of the vessel near the IPG. Arteries and veins tend to travel together along with a nerve fiber as they pass through the connective tissue and muscles. It is possible, therefore, that a concentration of current density where current passes out of the vessel may activate the adjacent nerve fiber. This can be prevented, however, by increasing the overall area for return of the current to the electrode as described previously.

Another explanation for how and where current density is sufficiently concentrated to generate pocket stimulation, however, relates to the formation of edge or corner effects along an edge of the conductive surface of an IPG. It is generally accepted that the current density is not uniformly distributed over the conductive surface of an IPG. Current density is higher along the edge(s), corner(s), and/or periphery of the uncoated surface of the IPG. This high or higher concentration of current density at specific locations on the outer surface of the IPG can significantly increase the likelihood of the patient experiencing pocket stimulation. One advantage of the IPGs 620 and 630 described above with reference to FIGS. 7A and 7B is that the insulating layers 624 and 634 are expected to inhibit corner or edge effects on the outer or periphery portions of the respective IPGs, and thereby inhibit and/or eliminate pocket stimulation during treatment.

FIGS. 8A and 8B illustrate an IPG 720 configured in accordance with yet another embodiment of the disclosure. FIG. 8A, for example, is a partially schematic, top plan view of the IPG 720. The IPG 720 includes a housing 722 having one or more conductive portions 724 and one or more insulating portions 726 arranged in a desired pattern on the housing 722 corresponding to a desired current return path across the housing 722. In other embodiments, the conductive portions 724 and insulating portions 726 can have a different arrangement relative to each other. The conductive portions 724 can be composed of a metal or other suitable conductive material. The insulating portions 726 can be composed of a plastic material, a silicon material, or another suitable insulating material. The width of the insulating portions 726 can vary depending on the configuration of the IPG and the desired treatment parameters. Further, in several embodiments one or more of the insulating portions 726 can have a different width (as shown in broken lines) than the other insulating portions 726 of the IPG 720.

FIG. 8B is a side cross-sectional view of the IPG 720 after implantation within a patient 800. As best seen in FIG. 8B, the individual insulating portions 726 project away from a surface of the housing 722 a desired distance D. The insulating portions 726 are accordingly stand-offs that space apart or separate the conductive portions 724 at the surface of the housing 722 from the patient's tissue 800. The distance D can vary between about 1 mm and about 3 mm. As discussed previously, spacing the patient's tissue apart from the conductive portions of the IPG can help reduce and/or eliminate pocket stimulation during treatment.

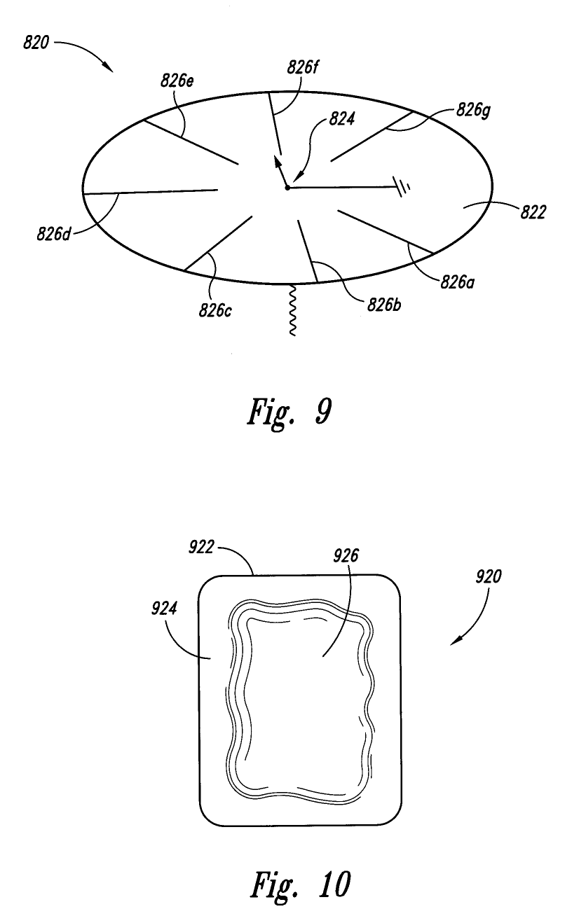

FIG. 9 is a schematic illustration of an IPG 820 configured in accordance with still another embodiment of the disclosure. The IPG 820 can have a plurality of separately activable portions at an outer surface of the IPG 820 generally similar to the IPGs 320 and 420 described above with reference to FIGS. 4-5C. The IPG 820 differs from the IPGs 320 and 420 described previously, however, in that the IPG 820 does not include conductive material disposed on the housing between the individual conductive portions. Rather, the IPG 820 can include a housing 822 composed of titanium or another suitable material that is a relatively poor electrical conductor. The IPG 820 includes a switch 824 positioned to activate/deactivate a plurality of leads 826 (seven are shown as leads 826a-g) extending across the housing 822. The switch 824 can include a software and/or a hardware switch.

The IPG 820 is configured to take advantage of the relatively poor conductivity of the titanium material in the housing 822. More specifically, during treatment, the entire housing 822 will be conductive, but current density will likely be the highest at the portions of the housing 822 proximate to the selected or activated leads 826a-g. If pocket stimulation occurs, one or more of the leads 826a-g can be deactivated, thus reducing current density at the respective portions of the housing 822.

FIG. 10 is a partially schematic illustration of an IPG 920 configured in accordance with yet another embodiment of the disclosure. The IPG 920 includes a housing 922 having an insulating portion 924 and a conductive portion 926. The IPG 920 differs from the IPGs described above in that the IPG 920 does not include a sharp line between the conductive and non-conductive portions on the IPG 920. Rather, there is a gradual transition between the insulating portion 924 and the conductive portion 926 on the housing 922, thus creating a conductivity “gradient” on the housing 922. In other embodiments, this gradient can be created by stippling the insulating material 924 or other suitable methods of creating a gradual transition between the insulating and conductive regions of the housing 922. In still other embodiments, the housing 922 may include multiple lines or regions of insulating material 924 on the IPG 920 to create a desired gradient. One particular aspect of this embodiment is that the conductivity gradient on the IPG 920 helps inhibit and/or eliminate the problems associated with “corner effects” described previously.

One feature of each of the embodiments described above with reference to FIGS. 4-10 is that each of the IPGs includes a housing with selectively activatable portions or regions that can be activated or deactivated during treatment to inhibit and/or eliminate pocket stimulation in a patient. Compared with conventional IPGs having merely coated or uncoated sides, the IPGs described above with reference to FIGS. 4-10 are expected to significantly reduce and/or eliminate pocket stimulation in patients during treatment.

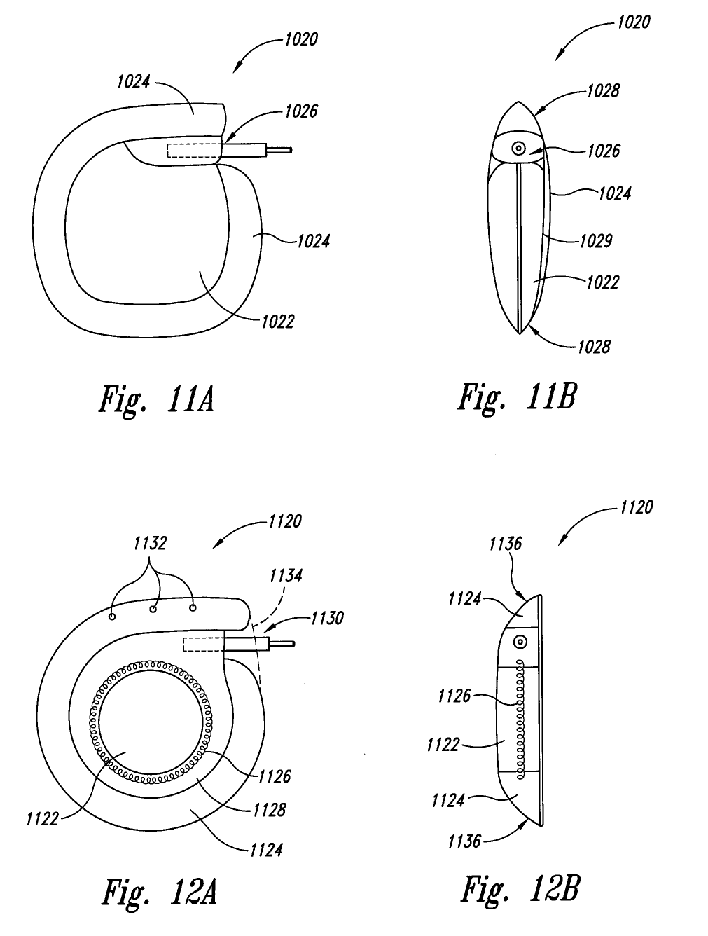

FIGS. 11A and 11B are partially schematic illustrations of an IPG 1020 configured in accordance with still another embodiment of the disclosure. FIG. 11A, for example, is a top plan view of the IPG 1020. The IPG 1020 includes a housing or enclosure 1022, a conformal portion 1024 around at least a portion of a periphery of the housing 1022, and an interconnect portion 1026. The housing 1022 can be composed of titanium or another suitable material. The conformal portion 1024 includes one or more molded portions around the periphery of the device. The conformal portion 1024 is generally flexible and can be composed of silicone (e.g., NuSil MED-4870, commercially available from NuSil Technology of Cupertino, Calif.), a combination of medical grade plastic material (e.g., Tecothane) overmolded with silicone, or another suitable material.

FIG. 11B is a side cross-sectional illustration of the IPG 1020. As best seen in FIG. 11B, the conformal portion 1024 of the IPG 1020 has tapered edges 1028. The tapered edges 1028 help transition the device into the patient's anatomy at the implant site and allow the IPG 1020 to be implanted with either side of the device facing away from the patient. In one particular aspect of this embodiment, the conformal portion 1024 includes a portion 1029 that encloses one full side of the IPG 1020. The portion 1029, for example, can provide a large bond surface to which the housing 1022 can be attached.

As described briefly above, current implantable medical devices (e.g., IPGs, pacemakers, neurostimulators, etc.) are generally constructed of a titanium enclosure to hermetically surround the components of the device and a generally rigid urethane header that houses the interconnections between the lead(s) and the device. These devices are generally planar and have a uniform thickness, with some rounding at the corner regions. This configuration is generally designed to simplify the manufacturing process. One problem with this conventional arrangement, however, is that such implantable devices generally do not conform to the patient's anatomy at the implant site and, accordingly, can be relatively uncomfortable for the patient.

One feature of the IPG 1020 is that the tapered edges 1028 reduce the thickness of the device at the periphery and provide a device geometry that more closely matches the patient anatomy at the implant site. Accordingly, the IPG 1020 is expected to be significantly more comfortable and cosmetically more acceptable for patients as compared to conventional IPGs having sharp corners and generally planar configurations.

FIGS. 12A and 12B are partially schematic illustrations of an IPG 1120 configured in accordance with still yet another embodiment of the disclosure. The IPG 1120 differs from the IPG 1020 described above in that the IPG 1120 has a different tapered profile and includes a number of different internal components. FIG. 12A, for example, is a top plan view of the IPG 1120. The IPG 1120 in this embodiment includes a housing or enclosure 1122, a conformal portion 1124 around at least a portion of the housing 1022, and an interconnect portion 1130. The housing 1122 can be composed of materials generally similar to the materials of the housing 1022 described above with reference to FIGS. 11A and 11B. The conformal portion 1124 includes one or more molded portions around the periphery of the housing 1122. The conformal portion 1124 can also be composed of materials generally similar to the conformal portion 1024 described previously. The conformal portion 1124 can further include one or more integral attachment (e.g., suture) points at desired locations. The attachment points can have a variety of different configurations and/or locations depending on the desired operational parameters for the IPG 1120.

The IPG 1120 may also include several additional internal components. For example, the IPG 1120 can include a coil 1126 and an integral header and charging coil portion 1128. The coil 1126 can be overmolded within the IPG 1120 and the header portion 1128 can be composed of a molded Tecothane component. Further, in several embodiments the IPG 1120 can include one or more sealing rings or portions 1134 (shown in broken lines) within the conformal portion 1124. The sealing rings 1134 can be integral with the material of the conformal portion 1124 rather than external to the interconnect portion 1130. In other embodiments, however, the IPG 1120 can have a different configuration and/or include different features.

FIG. 12B is a side cross-sectional illustration of the IPG 1120. Similar to the IPG 1020 described above, the conformal portion 1124 of the IPG 1120 also has tapered edges 1136 to help transition the device into the patient's anatomy. The tapered edges 1136 in this embodiment are optimized for one particular side of the IPG 1120. Accordingly, the non-tapered, generally planar side of the IPG 1120 is configured to face the patient (i.e., face posteriorly).

The IPGs 1020 and 1120 having tapered regions conforming more closely to the patient's anatomy described above with reference to FIGS. 11A-12B can also include one or more programmable regions as described above with reference to FIGS. 4-10. For example, the conformal portions 1024 and 1124 of the IPGs 1020 and 1120, respectively, can include activable portions that can be selectively activated/deactivated during treatment. The activatable portions can be on one or both sides of the corresponding IPGs.

The IPGs 1020 and 1120 can also have several other embodiments. For example, the tapered portions can have a variety of other configurations shaped to correspond to the patient and/or the anatomy of the implant site. Moreover, the various modules or components of the IPGs 1020 and 1120 can have a different arrangement relative to each other. Further, in several embodiments the tapered portions can be add-on components that are attached to existing planar IPGs. For example, one or more generally rigid or generally flexible tapered portions can be attached to a periphery of a non-conformal housing of an IPG. In still other embodiments, one or more components having a generally concave or convex profile can be attached to one or both sides of an IPG. The components could be specifically tailored to correspond to the patient's anatomy and the configuration of the IPG.

From the foregoing, it will be appreciated that specific embodiments of the disclosure have been described herein for purposes of illustration, but that various modifications may be made. For example, the IPGs described above may have configurations other than those shown in the Figures. Certain aspects of the disclosure described in the context of particular embodiments may be combined or eliminated in other embodiments. For example, any of the IPGs described herein can be placed in a resorbable or non-resorbable sheath as described above with reference to FIG. 3 before implantation. Moreover, as discussed above, the IPGs shown in FIGS. 4-11 can include conformal or tapered portions generally similar to those described with respect to FIGS. 11A-12B. Further, while advantages associated with certain embodiments have been described in the context of those embodiments, other embodiments may also exhibit such advantages, and not all embodiments need necessarily exhibit such advantages to fall within the scope of the disclosure. Accordingly, the disclosure is not limited except as by the appended claims.

I/We claim:

1. An implantable medical device for implantation in a patient, the implantable device comprising:
a signal generator positioned to be implanted in a patient, the signal generator including a housing and a plurality of selectively electrically activatable portions at an external surface of the housing;
a remote electrode device having at least one electrode positioned to be implanted beneath the patient's skull; and
a lead coupleable to the electrode device and the signal generator.

2. The implantable device of claim 1 wherein the selectively electrically activatable portions are arranged in a desired pattern at a major surface of the housing.

3. The implantable device of claim 1 wherein the selectively electrically activatable portions are arranged in a desired pattern along a periphery of the housing.

4. The implantable device of claim 1, further comprising a software switch configured to selectively activate and/or deactivate the corresponding activatable portions.

5. The implantable device of claim 1, further comprising a hardware switch configured to selectively activate and/or deactivate the corresponding activatable portions.

6. The implantable device of claim 1 wherein the individual activatable portions are electrically isolated from each other.

7. The implantable device of claim 1, further comprising a plurality of insulating portions on the housing and arranged in a desired pattern, and wherein the insulating portions separate the individual activatable portions from each other.

8. The implantable device of claim 7 wherein the plurality of selectively electrically activatable portions have an aggregate first surface area on the external surface and the insulating portions have an aggregate second surface area on the external surface, and wherein the first surface area is larger than the second surface area.

9. The implantable device of claim 7 wherein the insulating portions have an aggregate surface area on the external surface that is less than or approximately equal to an average surface area of each individual selectively electrically activatable portions.

10. The implantable device of claim 1 wherein the selectively activatable portions comprise discrete portions of conductive material on the housing.

11. The implantable device of claim 1 wherein the housing is composed of titanium, and wherein the selectively activatable portions comprise leads extending through the housing at least proximate to the external surface.

12. The implantable device of claim 1 wherein the housing includes a periphery portion having one or more tapered edges conforming at least in part to a geometry at an implant site of the patient.

13. The implantable device of claim 1 wherein the one or more tapered edges are integral components of the housing.

14. The implantable device of claim 1 wherein the one or more tapered edges are separate, discrete components configured to be attached to corresponding portions of the housing.

15. The implantable device of claim 1 wherein:
the housing comprises a first upper portion and a second lower portion spaced apart from each other and connected to each other via a transition region; and
the housing further comprises a first insulating layer disposed over the first upper portion and a second insulating layer disposed over the second lower portion, and wherein at least a portion of the transition region is not covered by the first or second insulating layers.

16. The implantable device of claim 1, further comprising a plurality of insulating portions on the housing positioned to separate the individual activatable portions from each other, and wherein the insulating portions project away from the external surface of the housing by a distance of from about 1 mm to about 3 mm.

17. The implantable device of claim 1 wherein the selectively electrically activatable portions are positioned to provide a set of electrical current return pathways during treatment signal delivery operations.

18. The implantable device of claim 1 wherein the signal generator is configured for monopolar activation.

19. The implantable device of claim 1 wherein the signal generator is positioned to be implanted below the patient's neck.

20. An implantable medical device for implantation in a patient, the implantable device comprising:
a signal generator positioned to be implanted in a patient;
a sheath surrounding the signal generator and spacing an external surface of the signal generator apart from tissue of the patient;
a remote electrode device having at least one electrode positioned to be implanted beneath the patient's skull; and
a lead coupleable to the electrode device and the signal generator.

21. The implantable device of claim 20 wherein the sheath is composed of a resorbable material.

22. The implantable device of claim 20 wherein the sheath is composed of collagen.

23. The implantable device of claim 20 wherein the sheath is composed of a non-resorbable material.

24. The implantable device of claim 20 wherein the sheath is composed of polyethylene terephthalate.

25. The implantable device of claim 20 wherein the sheath has a thickness of about 0.5 mm to about 5 mm.

26. The implantable device of claim 20 wherein the sheath is an integral component of the signal generator.

27. The implantable device of claim 20 wherein the sheath and signal generator are separate, discrete components.

28. The implantable device of claim 20 wherein the sheath is electrically conductive.

29. The implantable device of claim 20 wherein the sheath is composed of a porous material configured to be filled with a fluid before, during, or after implantation.

30. The implantable device of claim 29 wherein the fluid is an electrically conductive fluid disposed in the sheath before implantation.
